(19) United States
(12) Patent Application Publication
(10) Pub. No.: US 2017/0165280 A1
(43) Pub. Date: Jun. 15, 2017

(54) PHARMACEUTICAL FORMULATIONS FOR TREATING SKIN DISORDERS AND METHODS FOR FABRICATING AND USING THEREOF

(71) Applicant: Imprimis Pharmaceuticals, Inc., San Diego, CA (US)

(72) Inventor: Dennis Elias Saadeh, Irvine, CA (US)

(21) Appl. No.: 15/371,508

(22) Filed: Dec. 7, 2016

Related U.S. Application Data
(60) Provisional application No. 62/265,643, filed on Dec. 10, 2015.
(60) Provisional application No. 62/287,714, filed on Jan. 27, 2016.

(51) Int. Cl. A61K 31/65; A61K 9/48; A61K 9/00; A61K 31/202; A61K 31/455

(52) U.S. Cl. CPC A61K 31/65; A61K 31/202; A61K 31/455; A61K 9/0053; A61K 9/48

(57) ABSTRACT

Pharmaceutical compositions for treating, mitigating or preventing inflammatory skin diseases, disorders and/or pathologies are described, the compositions comprising a tetracycline-class antibiotic(s), omega fatty acid(s), and/or nicotinic acid or derivatives thereof. Methods for fabricating the compositions and using them are also described.

### CROSS-REFERENCE TO RELATED APPLICATIONS

This application claims priority under 35 U.S.C. §119(e) to each of the following U.S. Provisional applications: U.S. Ser. No. 62/265,643, filed on Dec. 10, 2015, and U.S. Ser. No. 62/287,714, filed on Jan. 27, 2016, each entitled “Pharmaceutical Formulations For Treating Skin Disorders and Methods for Fabricating and Using Thereof,” the entire contents of each of which is hereby incorporated by reference.

### FIELD OF THE INVENTION

The present invention relates generally to the field of dermatology and, more specifically, to compositions and methods designed to treat, mitigate or prevent inflammatory skin diseases, disorders and/or pathologies, and to methods of preparing such compositions.

### BACKGROUND

Many inflammatory skin disorders often result in painful and esthetically unattractive rashes, bumps, acne, and skin eruptions, such as pustules, nodules, macules, and the like. Among such disorders are various kinds of rosacea, acne, psoriasis, rhinophyma, a variety of types of dermatitis, etc. These disorders frequently cause a great deal of pain, discomfort, embarrassment and/or even disfigurement in some cases to those who suffer from them. These disorders are often very difficult to treat or prevent.

For example, in the case of rosacea, the symptoms include blushing, abnormal redness and irritation of the skin, and the appearance of visible red lines due to abnormal dilatation of capillary vessels. Other symptoms of rosacea include the formation of pimples (e.g., papules, nodules, or pustules), as well as the development of rhinophyma. In severe cases, rosacea can become irreversible and lead to permanent disfigurement.

Current pharmacological treatments include the use of antibiotics, vitamins, alpha-2 (α₂) adrenoceptors, and several kinds of non-steroidal anti-inflammatory agents. All such treatments are designed to control skin eruptions, inflammation and redness, but all are of limited effectiveness in many patients and can be typically used only for a limited amount of time due to frequent and sometimes severe side effects that would in many cases cause the discontinuation of treatment.

Accordingly, there exists a need for better methods and compositions for treatment, mitigation and/or prevention of inflammatory skin diseases, disorders and/or pathologies and their symptoms. This patent specification discloses such pharmaceutical compositions that would achieve positive patient outcomes while being free of drawbacks and deficiencies of existing formulations, and methods of fabricating and administering the same.

### SUMMARY

According to one embodiment of the invention, a pharmaceutical composition for treating, mitigating or preventing inflammatory skin diseases, disorders or pathologies is provided, the composition comprising a therapeutically effective quantity of at least one pharmaceutically acceptable anti-bacterial agent of the tetracycline class of broad-spectrum antibiotics or pharmaceutically suitable salts or hydrates thereof, and a therapeutically effective quantity of at least one pharmaceutically acceptable polyunsaturated fatty acid selected from the group of omega-3 fatty acids, omega-6 fatty acids, and omega-9 fatty acids; the composition is such that it does not form a foam.

According to other embodiments of the invention, the anti-bacterial agent is doxycycline or pharmaceutically suitable salts or hydrates thereof, and the polyunsaturated fatty acid is α-linolenic acid, eicosapentaenoic acid, docosahexaenoic acid, γ-linoleic acid, oleic acid, or a combination thereof.

According to yet another embodiment of the invention, a method for treating, mitigating or preventing inflammatory skin diseases, disorders or pathologies, comprising administering to a patient in need thereof a non-foaming pharmaceutical composition, in the form of a pill, a tablet or a troche, the composition comprising a therapeutically effective quantity of at least one pharmaceutically acceptable anti-bacterial agent of the tetracycline class of broad-spectrum antibiotics (such as, e.g., doxycycline) or pharmaceutically suitable salts or hydrates thereof, and a therapeutically effective quantity of at least one pharmaceutically acceptable polyunsaturated fatty acid from the group of omega-3 fatty acids, omega-6 fatty acids, and omega-9 fatty acids (such as, e.g., α-linolenic acid, eicosapentaenoic acid, docosahexaenoic acid, γ-linoleic acid, oleic acid).

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

The term “pharmaceutical composition” is defined as a chemical or biological compound or substance, or a mixture or combination of two or more such compounds or substances, intended for use in the medical diagnosis, cure, treatment, or prevention of disease or pathology.

The terms “anti-bacterial” and “antibiotic”, used herein interchangeably, refer to any substance or compound that destroys bacteria and/or inhibits the growth thereof via any mechanism or route.

The term “broad-spectrum antibiotics” refers to antibiotics that are effective against bacteria that give both a positive and a negative result in the Gram stain test.

The term “tetracycline class” refers to a group of broad-spectrum antibiotics of a polyketide class having an octahydrotetracene-2-carboxamide moiety having the following general structure:

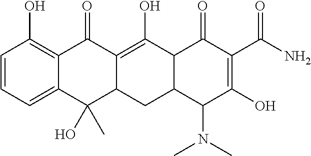 [SMILES: CN(C)C1C(O)=C(C(N)=O)C(=O)C2C(O)=C3C(=O)c4c(O)cccc4C(C)(O)C3CC21]

The term “doxycycline” (regular IUPAC name is 4-(dimethylamino)-3,5,10,12,12a-pentahydroxy-6-methyl-1,11-dioxo-1,4,4a,5,5a,6,11,12a-octahydrotetracene-2-carboxamide) is a chemical compound of the tetracycline class having the following chemical structure:

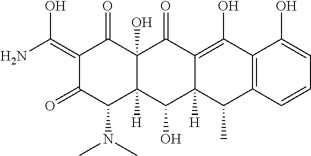 [SMILES: C[C@H]1c2cccc(O)c2C(O)=C2C(=O)[C@]3(O)C(=O)/C(=C(/N)O)C(=O)[C@@H](N(C)C)[C@@H]3[C@@H](O)[C@@H]21]

The term “nicotinic acid” (also known as vitamin B₃ or niacin) refers to a chemical compound with the regular IUPAC name pyridine-3-carboxylic acid and having the following chemical structure:

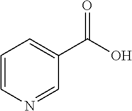

The term “derivatives of nicotinic acid” refers to such derivatives as those described below (i.e., niacinamide, nicotinamide, arecoline, nicorandil, nikethamide, nimodipine, trigonelline, ethionamide, iproniazid, isoniazid, and nialamide).

The terms “nicotinamide” and “niacinamide” refer interchangeably to a chemical compound with the regular IUPAC name pyridine-3-carboxamide and having the following chemical structure:

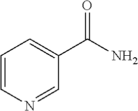

The term “polyunsaturated fatty acid” refers to an unsaturated fatty acid whose carbon chain has more than one double or triple bond.

The term “omega-3 fatty acid” (or “ω-3”) refers to a polyunsaturated fatty acid whose carbon chain has its first double bond at the third carbon atom from the methyl terminus of the chain; shown below as an illustration is the chemical structure of a commonly used ω-3 fatty acid, α-linoleic acid:

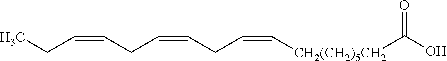

The term “omega-6 fatty acid” (or “ω-6”) refers to a polyunsaturated fatty acid whose carbon chain has its first double bond at the sixth carbon atom from the methyl terminus of the chain; shown below as an illustration is the chemical structure of a commonly used ω-6 fatty acid, γ-linoleic acid:

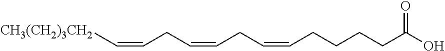

The term “omega-9 fatty acid” (or “ω-9”) refers to a polyunsaturated fatty acid whose carbon chain has its first double bond at the ninth carbon atom from the methyl terminus of the chain; shown below as an illustration is the chemical structure of a commonly used ω-9 fatty acid, oleic acid:

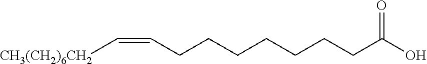

The terms “inflammatory skin diseases, disorders or pathologies” refer broadly to any skin disease, disorder or pathology characterized or caused by inflammation.

The term “rosacea” refers to a chronic skin condition that is characterized by blushing, abnormal redness and irritation of the skin, and the appearance of visible red lines as well as the formation of papules, nodules, or pustules, followed by possible development of rhinophyma.

The term “rhinophyma” refers to a skin condition cause by untreated rosacea which is characterized by prominent pores and thickening of the skin in the area of one's nose, giving it an unsightly appearance (ruddy and/or bulbous), often with papules.

The term “acne” refers to an inflammatory disease of the sebaceous glands, especially on the face, back, and chest, characterized by areas of blackheads, whiteheads, pimples and, in severe cases, by cysts and nodules, sometimes resulting in scarring.

The term “psoriasis” refers to a skin condition characterized by patches of red, scaly and itchy patches or spots and is used herein to be inclusive of all five known types of psoriasis (i.e., plaque, pustular, erythrodermic, guttate, and inverse).

The term “dermatitis” (also known as “eczema”) refers to a skin condition caused by inflammation and characterized by some or all of the following symptoms: redness, blistering, flaking, cracking, swelling, itching, dryness, crusting, and even bleeding.

For the purposes of the instant application, a “dry eye” disease, syndrome, or condition is considered as belonging to the group of inflammatory skin diseases, disorders or pathologies and is defined as one or several conditions associated with, or caused by, decreased tear production, increased tear film evaporation, or both, and characterized by redness, itching, and burning of the eye. Dry eye syndrome is inclusive of keratoconjunctivitis sicca.

The terms “tablet” and “pill” refer to a generally spherical (for pills) or disk-shaped (for tablets) compressed solid article containing a medicament to be taken orally. Included in these terms are softgels and liqui-gels, which are one-piece gelatin capsules surrounding a liquid medicament to be taken orally.

The term “troche” refers to a small tablet or lozenge (i.e., a medicated candy intended to be dissolved in the mouth), typically in a form of a disk, a ball or rhombic in cross-section, comprising medication and processed into a paste and dried.

The term “therapeutically effective amount” is defined as the amount of the compound or pharmaceutical composition that will elicit the biological or medical response of a tissue, system, animal or human that is being sought by the researcher, medical doctor or other clinician.

The terms “pharmaceutically acceptable” and “pharmaceutically suitable,” which are used herein interchangeably, are used to define as an active compound, an additive, a carrier, a diluent, a solvent, an excipient, or an ingredient of any other kind or type, as being compatible with the other ingredients of the formulation and not deleterious to the recipient thereof.

The terms “administration of a composition” or “administering a composition” are defined to include an act of providing a compound of the invention or pharmaceutical composition to the subject in need of treatment.

### B. Embodiments of the Invention

According to embodiments of the present invention, pharmaceutical compositions are provided for treating, mitigating or preventing inflammatory skin diseases, disorders or pathologies. The compositions of the present invention comprise therapeutically effective quantities of at least one pharmaceutically acceptable anti-bacterial agent or pharmaceutically suitable salts or hydrates thereof, and of:

(a) at least one pharmaceutically acceptable compound belonging to the group of polyunsaturated fatty acids, or

(b) at least one pharmaceutically acceptable compound belonging to the nicotinic acid family (e.g., nicotinic acid, derivatives of nicotinic acid, isonicotinic acid, and derivatives of isonicotinic acid), or

(c) any combination of compounds of groups (b) and (c).

It is further specifically provided that the compositions of the invention do not form a foam and are to be formulated as pills, tablets, capsules or troches for oral administration. So formulated compositions are then to be orally administered to a patient in need thereof for treating, mitigating or preventing inflammatory skin diseases, disorders or pathologies. Some specific, non-limiting examples of such diseases, disorders or pathologies to be treated include rosacea, acne, psoriasis, rhinophyma, dermatitis, and dry eye disease.

In some embodiments, anti-bacterial agent(s) that can be used in the compositions are broad-spectrum antibiotics of the tetracycline class such as doxycycline, tetracycline, minocycline, chlorotetracycline, demeclocycline, methacycline, oxytetracycline, demeclocycline, meclocycline, lymecyclinerolitetracycline, tigecycline or combinations thereof as well as pharmaceutically suitable salts or hydrates thereof. In one embodiment, the broad spectrum antibiotic is doxycycline or its salts or hydrates, such as doxycycline hyclate or doxycycline monohydrate.

The concentration of the anti-bacterial agent(s) in the compositions of the present application may be between about 3 mass % and about 75 mass % of the total mass of the pill, tablet, capsule or troche, such as between about 5 mass % and about 15 mass %, for example, about 10 mass %. In other words, for a typical pill, tablet, capsule or troche having the total mass of between 250 mg and about 1.5 g, the mass quantity of the anti-bacterial agent(s) may be between about 40 mg and about 200 mg, such as between about 40 mg and about 100 mg, for example about 70 mg.

In some embodiments, polyunsaturated fatty acid(s) that can be used in the compositions are omega-3 fatty acids, omega-6 fatty acids, omega-9 fatty acids or combinations thereof. Specific omega-3 fatty acids that can be used in some embodiments include α-linolenic acid, eicosatrienoic acid, eicosatetraenoic acid, eicosapentaenoic acid, docosapentaenoic acid, docosahexaenoic acid, hexadecatrienoic acid, tetracosapentaenoic acid, tetracosahexanoic acid, heneicosapentaenoic acid or stearidonic acid.

Specific omega-6 fatty acids that can be used in some embodiments include γ-linoleic acid, adrenic acid, arachidonic acid, calendic acid, docosadienoic acid or eicosadienoic acid. Specific omega-9 fatty acids that can be used in some embodiments include oleic acid, erucic acid, mead acid, nervonic acid, elaidic acid or gondoic acid.

Combinations of omega-3 fatty acids, omega-6 fatty acids and/or omega-9 fatty acids that may prove to be useful include α-linolenic acid (ω-3), eicosapentaenoic acid (ω-3) or docosahexaenoic acid (ω-3), γ-linoleic acid (ω-6) and/or oleic acid (ω-9).

The concentration of the polyunsaturated fatty acids(s) in the compositions of the present application may be, for omega-3 acids only, between about 5 mass % and about 65 mass %, of the total mass of the pill, tablet, capsule or troche, such as between about 5 mass % and about 30 mass %, for example, about 15 mass %. In other words, for a typical pill, tablet, capsule or troche having the total mass of between 250 mg and about 1.5 g, the mass quantity of the omega-3 acid(s) may be between about 200 mg and about 1 g, such as between about 100 mg and about 500 mg, for example about 250 mg. If omega-6 and/or omega-9 acid(s) are used (whether in addition to, or instead of, omega-3 acid(s)), their mass quantities in the pill, tablet, capsule or troche may be between about one-half and one-third of the quantities of the omega-3 acids mentioned above.

In some embodiments, the nicotinic acid family compound(s) include nicotinic acid, isonicotinic acid niacinamide, nicotinamide, arecoline, nicorandil, nikethamide, nimodipine, trigonelline, ethionamide, iproniazid, isoniazid, and nialamide.

The concentration of the nicotinic or isonicotinic acid-based compound(s) in the compositions of the present application may be between about 5 mass % and about 50 mass % of the total mass of the pill, tablet, capsule or troche, such as between about 15 mass % and about 45 mass %, for example, about 40 mass %. In other words, for a typical pill, tablet, capsule or troche having the total mass of between 400 mg and about 650 mg, the mass quantity of the anti-bacterial agent(s) may be between about 100 mg and about 300 mg, such as, for example about 200 mg.

The pharmaceutical formulations that are described herein may, in addition, optionally contain other pharmacologically active compounds such as at least one antifungal medicament, in the quantity of about 10 mg per pill, tablet, capsule or troche. Those having ordinary skill in the art can determine what specific antifungal medicaments are to be used, if any. Non-limiting examples of the antifungal medicaments that may be used are ketoconazole and fluconazole.

As mentioned above, the pharmaceutical composition that is the subject matter of the instant application may further optionally include one or several pharmaceutically acceptable excipient(s). In some embodiments, an excipient that can be used may be one or several filler(s) to be selected by those having ordinary skill in the art, such as microcrystalline cellulose and/or hydroxypropyl methylcellulose (e.g., Methocell® E4M available from Dow Chemical Co. of Midland, Mich.). For example, as is known in the art, Methocell® E4M can be used for preparing the formulations in the form of AR (i.e., acid-resistant) capsules to protect from gastric acid and delay dissolution.

According to further embodiments, methods for fabricating the above-described pharmaceutical compositions are provided. A one-batch formulation method may be used, where the components of the pharmaceutical formulation can be combined in a single container; the components may be added to the container simultaneously or consecutively. In one exemplary, non-limiting procedure, a quantity of anti-bacterial agent(s) and a quantity of polyunsaturated fatty acids(s) may be placed into a mixing container (e.g., a mortar) followed by dry mixing with a pestle. The resulting product may then be adapted for oral administration, for example formulated and shaped as pills, tablets or capsules according to methods known to those having ordinary skill in the art.

It will be understood by those having ordinary skill in the art that the specific dose levels and frequency of administration for any particular patient may be varied and will depend upon a variety of factors including the activity of the specific compound employed, the metabolic stability and length of action of that compound, the age, body weight, general health, gender, diet, and the severity of the particular inflammatory skin disease, disorder or pathology being treated.

In additional embodiments, pharmaceutical kits are provided. The kit includes a sealed container approved for the storage of pharmaceutical compositions, and the above-described pharmaceutical composition. An instruction for the use of the composition and any information about the composition are to be included in the kit.

The following examples are provided to further elucidate the advantages and features of the present invention, but are not intended to limit the scope of the invention. The examples are for the illustrative purposes only. USP pharmaceutical grade products were used in preparing the formulations described below.

### Example 1. Preparing a Pharmaceutical Composition No. 1

A pharmaceutical composition was prepared as described below. The following products were used in the amounts and concentrations specified:

(1) about 4.0 g of doxycycline hyclate powder having 0.04 g of the active ingredient, doxycycline hyclate;

(2) about 20.0 g of omega-3 acid/omega-6 acid powder (Kirkman's EFA™ powder) having 0.2 g of the active ingredient, omega-3 acid/omega-6 acid; and

(3) about 100.0 g of capsules, AR Caps® Clear, Size 0.

Doxycycline hyclate powder and Kirkman's EFA™ powder were mixed using a mortar a pestle method by using the principles of trituration and geometric dilution known to those having the skill in the art of preparing pharmaceutical compositions. To wit, Kirkman's EFA™ powder was mixed into doxycycline hyclate powder in small portions until the former was completely mixed into the latter.

The resulting product was encapsulated into AR Capsules, the capsules were put into an airtight container, and the container was labeled accordingly.

### Example 2. Preparing a Pharmaceutical Composition No. 2

A pharmaceutical composition was prepared as described below. The following products were used in the amounts and concentrations specified:

(1) about 10.0 g of doxycycline hyclate powder having 0.1 g of the active ingredient, doxycycline hyclate;

(2) about 25.0 g of omega-3 acid/omega-6 acid powder (Kirkman's EFA™ powder) having 0.25 g of the active ingredient, omega-3 acid/omega-6 acid mixture; and

(3) about 100.0 g of capsules, AR Caps® Clear, Size 0.

The procedure described in Example 1 was used for preparing the composition.

### Example 3. Preparing a Pharmaceutical Composition No. 3

A pharmaceutical composition was prepared as described below. The following products were used in the amounts and concentrations specified:

(1) about 100.0 mg of doxycycline hyclate powder having 1.0 mg of the active ingredient, doxycycline hyclate;

(2) about 300.0 mg of niacinamide; and

(3) about 215.0 mg of Methocel K100 capsules, AR Caps® Clear, Size 0.

The procedure described in Example 1 was used for preparing the composition.

### Example 4. Preparing a Pharmaceutical Composition No. 4

A pharmaceutical composition was prepared as described below. The same products in the same quantities as those described in Example 3, above, were used, except doxycycline hyclate used in Example 3 was replaced with the same amount of minocycline. The procedure described in Example 1 was used for preparing the pharmaceutical composition.

### Example 5. Preparing a Pharmaceutical Composition No. 5

A pharmaceutical composition was prepared as described below. The following products were used in the amounts and concentrations specified:

(1) about 100.0 mg of doxycycline hyclate powder having 1.0 mg of the active ingredient, doxycycline hyclate;

(2) about 150.0 mg of omega-3 acid/omega-6 acid powder (Kirkman's EFA™ powder) having 1.5 mg of the active ingredient, omega-3 acid/omega-6 acid mixture; and

(3) about 100.0 mg of niacinamide; and

(4) about 215.0 mg of Methocel K100 capsules, AR Caps® Clear, Size 0.

The procedure described in Example 1 was used for preparing the composition.

### Example 6. Preparing a Pharmaceutical Composition No. 6

A pharmaceutical composition was prepared as described below. The same products in the same quantities as those described in Example 5, above, were used, except doxycycline hyclate used in Example 5 was replaced with the same amount of minocycline. The procedure described in Example 1 was used for preparing the composition.

Although the invention has been described with reference to the above examples, it will be understood that modifications and variations are encompassed within the spirit and scope of the invention. Accordingly, the invention is limited only by the following claims.

What is claimed is:

1. A pharmaceutical composition for treating, mitigating or preventing inflammatory skin diseases, disorders or pathologies, the composition comprising:
(a) a therapeutically effective quantity of at least one pharmaceutically acceptable anti-bacterial agent independently selected from the group consisting of broad-spectrum antibiotics of the tetracycline class; and
(b) a therapeutically effective quantity of at least one pharmaceutically acceptable compound selected from:
(b1) polyunsaturated fatty acids independently selected from the group consisting of omega-3 fatty acids, omega-6 fatty acids, omega-9 fatty acids; and
(b2) a family of nicotinic acids selected from the group consisting of nicotinic acid, derivatives of nicotinic acid, isonicotinic acid, and derivatives of isonicotinic acid,
wherein the composition does not form a foam and is formulated in a form that is suitable for oral administration.

2. The composition of claim 1, wherein the anti-bacterial agent is selected from the group consisting of doxycycline, tetracycline, minocycline, chlorotetracycline, demeclocycline, methacycline, oxytetracycline, demeclocycline, meclocycline, lymecyclinerolitetracycline, tigecycline, pharmaceutically suitable salts thereof, and hydrates thereof.

3. The composition of claim 2, wherein the anti-bacterial agent is selected from the group consisting of doxycycline, doxycycline hyclate, and doxycycline monohydrate.

4. The composition of claim 1, wherein the omega-3 fatty acid is selected from the group consisting of α-linolenic acid, eicosatrienoic acid, eicosatetraenoic acid, eicosapentaenoic acid, docosapentaenoic acid, docosahexaenoic acid, hexadecatrienoic acid, tetracosapentaenoic acid, tetracosahexanoic acid, heneicosapentaenoic acid, and stearidonic acid.

5. The composition of claim 1, wherein the omega-6 fatty acid is selected from the group consisting of γ-linoleic acid, adrenic acid, arachidonic acid, calendic acid, docosadienoic acid, and eicosadienoic acid.

6. The composition of claim 1, wherein the omega-9 fatty acid is selected from the group consisting of oleic acid, erucic acid, mead acid, nervonic acid, elaidic acid, and gondoic acid.

7. The composition of claim 3, wherein the polyunsaturated fatty acids are selected from the group consisting of α-linolenic acid, eicosapentaenoic acid, docosahexaenoic acid, γ-linoleic acid, oleic acid, and combinations thereof.

8. The composition of claim 1, wherein the derivatives of nicotinic acid are independently selected from the group consisting of niacinamide, nicotinamide, arecoline, nicorandil, nikethamide, nimodipine, trigonelline, ethionamide, iproniazid, isoniazid, and nialamide.

9. The composition of claim 8, wherein the derivative of nicotinic acid is niacinamide.

10. The composition of claim 1, wherein the composition is in the form of a pill, tablet, capsule or troche.

11. A method for treating, mitigating or preventing inflammatory skin diseases, disorders or pathologies, comprising administering to a patient in need thereof a pharmaceutical composition, comprising:
(a) a therapeutically effective quantity of at least one pharmaceutically acceptable anti-bacterial agent independently selected from the group consisting of broad-spectrum antibiotics of the tetracycline class;
(b) a therapeutically effective quantity of at least one pharmaceutically acceptable compound selected from:
(b1) polyunsaturated fatty acids independently selected from the group consisting of omega-3 fatty acids, omega-6 fatty acids, omega-9 fatty acids; and
(b2) a family of nicotinic acids selected from the group consisting of nicotinic acid, derivatives of nicotinic acid, isonicotinic acid, and derivatives of isonicotinic acid,
wherein the composition does not form a foam and is formulated in a form that is suitable for oral administration.

12. The method of claim 11, wherein the anti-bacterial agent is selected from the group consisting of doxycycline, tetracycline, minocycline, chlorotetracycline, demeclocycline, methacycline, oxytetracycline, demeclocycline, meclocycline, lymecyclinerolitetracycline, tigecycline, pharmaceutically suitable salts thereof, and hydrates thereof.

13. The method of claim 11, wherein the anti-bacterial agent is selected from the group consisting of doxycycline, doxycycline hyclate, and doxycycline monohydrate.

14. The method of claim 11, wherein the omega-3 fatty acid is selected from the group consisting of α-linolenic acid, eicosatrienoic acid, eicosatetraenoic acid, eicosapentaenoic acid, docosapentaenoic acid, docosahexaenoic acid, hexadecatrienoic acid, tetracosapentaenoic acid, tetracosahexanoic acid, heneicosapentaenoic acid, and stearidonic acid.

15. The method of claim 11, wherein the omega-6 fatty acid is selected from the group consisting of γ-linoleic acid, adrenic acid, arachidonic acid, calendic acid, docosadienoic acid, and eicosadienoic acid.

16. The method of claim 11, wherein the omega-9 fatty acid is selected from the group consisting of oleic acid, erucic acid, mead acid, nervonic acid, elaidic acid, and gondoic acid.

17. The method of claim 11, wherein the polyunsaturated fatty acids are selected from the group consisting of α-linolenic acid, eicosapentaenoic acid, docosahexaenoic acid, γ-linoleic acid, and oleic acid.

18. The method of claim 11, wherein the derivatives of nicotinic acid are independently selected from the group consisting of niacinamide, nicotinamide, arecoline, nicorandil, nikethamide, nimodipine, trigonelline, ethionamide, iproniazid, isoniazid, and nialamide.

19. The method of claim 11, wherein the composition is administered orally in the form of a pill, tablet or troche.

20. The method of claim 11, wherein the disease, disorder or pathology is selected from the group consisting of rosacea, acne, psoriasis, rhinophyma, dermatitis, and dry eye disease.
